(19) United States
(12) Patent Application Publication
(10) Pub. No.: US 2025/0114329 A1
(43) Pub. Date: Apr. 10, 2025

(54) METHODS FOR TREATING AML-MRC AND MDS

(71) Applicants: Ascentage Pharma (Suzhou) Co., LTD, Suzhou (CN); Ascentage Pharma Group Corp Limited, Hong Kong (CN)

(72) Inventors: Dajun YANG, Suzhou (CN); Yifan ZHAI, Suzhou (CN); Guangfeng WANG, Suzhou (CN); Ping MIN, Suzhou (CN)

(21) Appl. No.: 18/727,848

(22) PCT Filed: Jan. 11, 2023
(86) PCT No.: PCT/CN2023/071797

§ 371 Date: Jul. 10, 2024

(30) Foreign Application Priority Data
Jan. 11, 2022 (WO) PCT/CN2022/071343
Nov. 2, 2022 (WO) PCT/CN2022/129292

(51) Int. Cl. A61K 31/407; A61K 31/437; A61K 31/454; A61K 31/706; A61K 31/7068; A61K 45/06

(52) U.S. Cl. CPC A61K 31/407; A61K 31/437; A61K 31/454; A61K 31/706; A61K 31/7068; A61K 45/06

(57) ABSTRACT

Provided are methods for treating hematologic malignancy, especially AML-MRC, MDS and/or MM. Specifically, provided are methods for treating AML-MRC and MDS with a compound of formula (I) or a pharmaceutically acceptable salt thereof, optionally in combination with at least one additional anti-cancer agent such as 5-azacitidine or cytarabine, and treating MM with a compound of formula (I) or a pharmaceutically acceptable salt thereof, optionally in combination with at least one additional anti-cancer agent such as pomalidomide, thalidomide, lenalidomide, and/or dexamethasone.

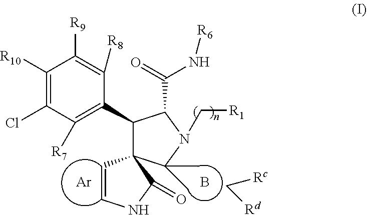

The present invention claims the priority of the PCT/CN2022/071343, filed on Jan. 11, 2022, and the priority of the PCT/CN2022/129292, filed on Nov. 2, 2022, the contents of which are incorporated herein by its entirety.

### FIELD OF THE INVENTION

The present disclosure relates to methods for treating hematologic malignancy, especially acute myeloid leukemia (AML) with myelodysplasia-related changes (AML-MRC) and/or myelodysplastic syndrome (MDS) and/or multiple myeloma (MM).

### BACKGROUND OF THE INVENTION

AML is a hematologic malignancy involving unregulated proliferation of myeloid precursor cells in the bone marrow (BM) and peripheral blood (PB), resulting in unrestrained propagation of immature myeloblasts that disrupts normal hematopoiesis. AML is a highly heterogeneous neoplasm with diverse biology and clinical outcomes based on specific characteristics of leukemia cells. The prognosis of patients with AML varies based upon clinical characteristics including age, fitness for intensive chemotherapy (IC), antecedent hematologic disorders (AHD), and leukemia-associated risk factors such as cytogenetics and genetic mutations.

AML-MRC is a distinct biologic subtype of AML. AML-MRC is defined by the World Health Organization (WHO) in 2008 by the presence of multilineage dysplasia (MLD), and/or myelodysplastic syndrome (MDS)-related cytogenetics, and/or a history of MDS or MDS/myeloproliferative neoplasm (MPN). In the 2016 WHO classification, AML-MRC was preserved as a distinct entity, with some minor revisions in MDS-related cytogenetics. Most studies have reported that the prognosis of AML-MRC was worse than that of AML not otherwise specified (NOS). There is a high unmet need to develop novel therapeutic options for AML-MRC.

Myelodysplastic syndromes (MDS) are defined by dysplasia and ineffective hematopoiesis leading to cytopenias and risk of evolution to acute myeloid leukemia (AML).

Currently, few therapies are approved for MDS, which include the oral immunomodulatory drug lenalidomide and two hypomethylating agents (HMAs) azacitidine and decitabine. The dire need for new therapies is evident as these patients will ultimately have disease progression with a lack of therapeutic options in the relapse/refractory setting.

Multiple myeloma (MM) is a hematological malignancy affecting the plasma cells. It is a malignant neoplasm of B-cells characterized by the uncontrolled proliferation of plasma cells in the bone marrow. Despite the advancements in MM treatment, it has a poor prognosis with a median survival of 3-5 years. There is an unmet need for effective treatments for multiple myeloma (MM).

### BRIEF DESCRIPTION OF DRAWINGS

FIG. 1A-IF show the synergistic anti-proliferation effects of alrizomadlin (Compound C) and IMiDs (pomalidomide and lenalidomide) in MM MMIS, MOLP-8, and H929 cell lines. Combination index (CI) was calculated using CalcuSyn (Biosoft) software.

CI value <1.0 indicates synergism. Herein, FIGS. 1A, 1i, and 1C show alrizomadlin, pomalidomide and their combination in MM MM1S, MOLP-8, and H929 cell lines, respectively; FIGS. 1D, 1E, and 1F show alrizomadlin, lenalidomide and their combination in MM MM1S, MOLP-8, and H929 cell lines, respectively.

FIGS. 2A and 2B show the synergistic anti-proliferation effects of alrizomadlin and pomalidomide induced cell cycle arrest and apoptosis of NCI-H929 cells, respectively. Cells were treated with Alrizomadlin or in combination with pomalidomide at indicated concentrations for 24 hrs, FACS was used to determining the cell cycle arrest and apoptosis.

FIG. 3A-3C show the potential mechanism of action for synergy of alrizomadlin with pomalidomide in H929 cells. Herein, FIG. 3A shows Alrizomadlin and pomalidomide induced degradation of transcription factors IKZF-1/IKZF-3, IRF4 and cMYC, inhibited phosphorylation of downstream AKT and ERK; FIG. 3B shows Alrizomadlin upregulated P53, MDM2 and P21, and when combined with pomalidomide additionally inhibited phosphorylation of Rb and promoted expression of BIM; FIG. 3C shows Alrizomadlin and pomalidomide induced upregulation of P21 and cleavage of CASPASE-3 and PARP-1. Cells were treated for 24 hrs or 48 hrs, Western Blotting assay was performed to detecting the expression of proteins.

FIG. 4A-4D show the synergistic effects of alrizomadlin and pomalidomide in NCI H929 and MOLP-8 xenograft models in nude mice. Herein FIGS. 4A and 4C show Tumor volume in NCI H929 and MOLP-8 xenograft models, respectively; FIGS. 4B and 4D show Tumor weight change in NCI H929 and MOLP-8 xenograft models, respectively. DXM, dexamethasone; PO, oral administration; IV, injection of vein; QD, once a day; BIW, twice a week; W, week; T/C %, Tumor growth rate; Synergy score was calculated using Clarke's method; score >1 indicates synergism.

### SUMMARY OF THE INVENTION

In one aspect, the present disclosure provides a method for the treatment of a disease in a subject in need thereof, comprising administering to the subject a therapeutically effective amount of a compound of formula I or a pharmaceutically acceptable salt thereof; wherein the disease is hematologic malignancy; especially acute myeloid leukemia with myelodysplasia-related changes (AML-MRC) and/or myelodysplastic syndrome (MDS) and/or multiple myeloma (MM); for example acute myeloid leukemia with myelodysplasia-related changes (AML-MRC) or myelodysplastic syndrome (MDS);

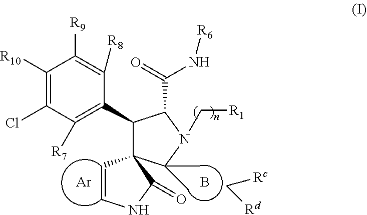

wherein,

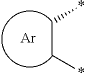

is selected from the group consisting of

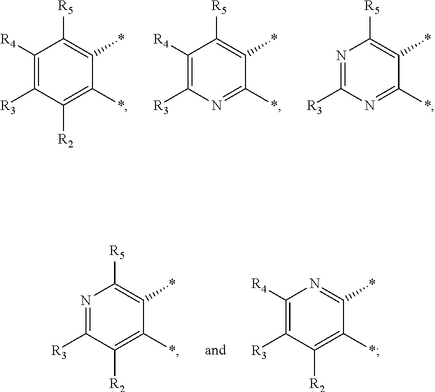

ring B is a C₄₋₇ carbocyclic ring;
R₁ is H, substituted or unsubstituted C₁₋₄ alkyl, substituted or unsubstituted C₃₋₈ cycloalkyl, substituted or unsubstituted heterocycloalkyl, OR^{a}, or NR^{a}R^{b};
n is 0, 1, or 2;
R₂, R₃, R₄, R₅, R₇, R₈, R₉, and R₁₀ are independently selected from the group consisting of H, F, Cl, CH₃ and CF₃;
R₆ is

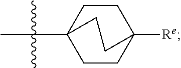

each of R^{a} is independently H, or substituted or unsubstituted C₁₋₄ alkyl;
R^{b} is H, or substituted or unsubstituted C₁₋₄ alkyl;
R^{c} and R^{d} are substituents on one carbon atom of ring B, wherein
R^{c} is H, C₁₋₃ alkyl, C₁₋₃ alkylene-OR^{a}, OR^{a}, or halogen;
R^{d} is H, C₁₋₃ alkyl, C₁₋₃ alkylene-OR^{a}, OR^{a}, or halogen;
or, R^{c} and R^{d} are taken together with the carbon to which they are attached to form a 4 to 6-membered spiro substituent optionally containing oxygen atom or nitrogen atom;
R^{e} is —C(═O)OR^{a}, —C(═O)NR^{a}R^{b}, or —C(═O)NHSO₂CH₃.

In some embodiments, the compound of formula I is preferably alrizomadlin.

In some embodiments, the present disclosure provides a method for the treatment of a disease in a subject in need thereof, comprising administering to the subject a therapeutically effective amount of a compound of formula I or a pharmaceutically acceptable salt thereof and one or more anticancer reagents.

In some embodiments, the present disclosure provides a method for the treatment of a disease in a subject in need thereof, comprising administering to the subject a therapeutically effective amount of a compound of formula I or a pharmaceutically acceptable salt thereof and one or more anticancer reagents for treating or suppressing a cancer, reducing its severity, lowering its risk or inhibiting its metastasis in an individual.

In another aspect, the invention provides the use of a therapeutically effective amount of a compound of formula I or a pharmaceutically acceptable salt thereof for treating or suppressing a cancer, reducing its severity, lowering its risk or inhibiting its metastasis in an individual.

In another aspect, the present disclosure provides a use of the compound of formula I or a pharmaceutically acceptable salt thereof in the manufacture of a medicament for the treatment of a disease, wherein the disease is hematologic malignancy; especially AML-MRC, MDS or MM, for example AML-MRC or MDS.

In some embodiments, the invention provides the use of a therapeutically effective amount of a compound of formula I and one or more anticancer reagents for treating or suppressing a cancer, reducing its severity, lowering its risk or inhibiting its metastasis in an individual.

In another aspect, the present disclosure provides a use of a therapeutically effective amount of the compound of formula I or a pharmaceutically acceptable salt thereof and one or more anticancer reagents in the manufacture of a medicament for the treatment of a disease, wherein the disease is hematologic malignancy; especially AML-MRC, MDS or MM, for example AML-MRC or MDS.

In another aspect, the invention provides a pharmaceutical combination for treating or suppressing a cancer, reducing its severity, lowering its risk or inhibiting its metastasis in an individual comprising a compound of formula I or a pharmaceutically acceptable salt thereof and one or more anticancer reagents.

In another aspect, the invention provides a pharmaceutical combination for the treatment of a disease, wherein the disease is hematologic malignancy; especially AML-MRC, MDS or MM, for example AML-MRC or MDS comprising a compound of formula I or a pharmaceutically acceptable salt thereof and one or more anticancer reagents.

In another aspect, the present disclosure provides a pharmaceutical composition for the treatment of a disease, comprising the compound of formula I or a pharmaceutically acceptable salt thereof and a pharmaceutical acceptable excipient, wherein the disease is AML-MRC, MDS or MM, for example AML-MRC or MDS.

In another aspect, the present disclosure provides a pharmaceutical composition for treating or suppressing a cancer, reducing its severity, lowering its risk or inhibiting its metastasis in an individual, comprising the compound of formula I or a pharmaceutically acceptable salt thereof and one or more additional anticancer reagents.

In some embodiments, the disease is AML-MRC.

In some embodiments, the disease is MDS.

In some embodiments, the disease is MM.

In some embodiments, the anti-cancer agent is antimetabolite (e.g., 5-azacitidine or cytarabine).

In some embodiments, the anticancer reagent is 5-azacitidine.

In some embodiments, the anticancer reagent is dexamethasone.

In some embodiments, the anticancer reagents are immuno-modulatory drugs.

In some embodiments, the anticancer reagent is pomalidomide, thalidomide or lenalidomide.

In some embodiments, the anticancer reagent is pomalidomide or Lenalidomide.

In some embodiments, the anticancer reagent is pomalidomide.

### DETAILED DESCRIPTION OF THE INVENTION

Unless defined otherwise, all technical and scientific terms used herein have the same meaning as commonly understood by one of ordinary skill in the art to which this disclosure belongs.

The term “comprises” refers to “includes, but is not limited to”.

As used herein, the term “treating”, and “treatment” refer to therapeutic therapy. When referring to a particular condition, the treatment refers to: (1) alleviating one or more of the biological manifestations of a disease or a condition, (2) interfering with (a) one or more points in the biological cascade that leads to a condition or (b) one or more of the biological manifestations of a condition, (3) improving one or more of symptoms, effects or side effects associated with a condition or one or more of the symptoms, effects or side effects associated with a condition or treatment thereof, or (4) slowing the progression of one or more of the biological manifestations of a disorder or a condition.

As used herein, the term “therapeutically effective amount” refers to an amount that is effective to elicit the desired biological or medical response, including the amount of a compound that, when administered to a subject for treating a disorder, is sufficient to effect such treatment of the disorder. The therapeutically effective amount will vary depending on the disorder, and its severity, and the age, weight, etc. of the subject to be treated. The therapeutically effective amount may be in one or more doses (for example, a single dose or multiple doses may be required to achieve the desired treatment endpoint). A therapeutically effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the term “subject” to which administration is contemplated includes any animal (e.g., humans).

As used herein, the term “pharmaceutically acceptable” as used herein refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of compound 1 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

As used herein, the use of the terms “a”, “an”, “the”, and similar referents in the context of describing the present disclosure (especially in the context of the claims) are to be construed to cover both the singular and the plural, unless otherwise indicated. The use of any and all examples, or exemplary language (including “e.g.”, “such as” and “for example”) provided herein, is intended to better illustrate the present disclosure and is not a limitation on the scope of the present disclosure unless otherwise claimed. No language in the specification should be construed as indicating any non-claimed element as essential to the practice of the present disclosure.

As used herein, the term “dose” is an amount of the certain substance to be taken at one time.

As used herein, the term “alkyl” refers to a linear or branched saturated hydrocarbyl group having an indicated number of carbon atoms, including but not limited to methyl, ethyl, n-propyl, iso-propyl, n-butyl, sec-butyl, tert-butyl, n-pentyl, 2-methylbutyl, 3-methylbutyl, 2,2-dimethylpropyl, n-hexyl, 2-methylpentyl, 3-methylpentyl, 4-methylpentyl, 2,2-dimethylbutyl, 2,3-dimethylbutyl, 3,3-dimethylbutyl, and 2-ethybutyl. The term Cm-n means the alkyl group has “m” to “n” carbon atoms.

The term “alkylene” refers to an alkyl group having a substituent. When an alkyl group is indicated to be substituted by an indicated substituent (e.g., substituted alkyl, alkyl substituted by), the alkyl group can be substituted by the indicated substituent. The alkyl can be substituted by one or more (e.g., one to three) of the group independently selected from halogen, trifluoromethyl, trifluoromethoxy, hydroxyl, alkoxyl, nitro, cyano, alkylamino, and amino groups.

As used herein, the term “halogen” is defined as fluorine, chlorine, bromine, and iodine.

As used herein, the term “hydroxyl” is defined as —OH.

As used herein, the term “alkoxyl” is defined as —OR, wherein R is alkyl.

As used herein, the term “amino” is defined as —NH₂, and the term “alkylamino” is defined as —N(R)₂, wherein at least one R is alkyl and the second R is alkyl or hydrogen.

As used herein, the term “carbamoyl” is defined as —C(═O)N(R)₂.

As used herein, the term “carboxy” is defined as —C(═O)OH or a salt thereof.

As used herein, the term “nitro” is defined as —NO₂.

As used herein, the term “cyano” is defined as —CN.

As used herein, the term “trifluoromethyl” is defined as —CF₃.

As used herein, the term “trifluoromethoxy” is defined as —OCF₃.

As used herein, the term “aryl” refers to a monocyclic or polycyclic aromatic group, preferably a monocyclic or bicyclic aromatic group. Examples of aryl groups include, but are not limited to, phenyl, naphthyl, fluorenyl, azulenyl, anthryl, phenanthryl, pyrenyl, biphenyl, and terphenyl. Aryl also refers to bicyclic and tricyclic carbon rings, where one ring is aromatic and the others are saturated, partially unsaturated, or aromatic, for example, dihydronaphthyl, indenyl, indanyl, or tetrahydronaphthyl (tetralinyl). Unless otherwise indicated, an aryl group can be unsubstituted or substituted with one or more, and in particular one to four, of the group independently selected from, for example, halogen, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “heterocyclic” refers to a heteroaryl and heterocycloalkyl ring systems.

As used herein, the term “heteroaryl” refers to a monocyclic or bicyclic ring system containing one or two aromatic rings and containing at least one nitrogen, oxygen, or sulfur atom in an aromatic ring. Each ring of a heteroaryl group can contain one or two 0 atoms, one or two S atoms, and/or one to four N atoms, provided that the total number of heteroatoms in each ring is four or less and each ring contains at least one carbon atom. In certain embodiments, the heteroaryl group has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl.

Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quiazolinyl, thiadiazolopyrimidyl, and thienopyridyl. Unless otherwise indicated, a heteroaryl group can be unsubstituted or substituted with one or more, and in particular one to four, of the group independently selected from, for example, halogen, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

In certain embodiments, the heteroaryl is a stable monocyclic ring with up to six atoms or a stable bicyclic ring in which each ring contains up to six atoms wherein at least one of the rings is an aromatic ring having 1 to 4 heteroatom(s) independently selected from the group consisting of nitrogen, oxygen and sulfur. Any N-oxidation derivatives of the heteroaryl containing nitrogen atoms are also included in the definition of heteroaryl. When heteroaryl group as a substituent is a bicyclic ring and one of the two rings is non-aromatic or without heteroatom, this bicyclic ring is fused through the aromatic ring or the ring containing heteroatom.

As used herein, the term “cycloalkyl” refers to a monocyclic or bicyclic, saturated or partially unsaturated cyclic hydrocarbyl group having an indicated number (e.g., 3 to 8) of ring carbon atoms, including but is not limited to cyclopropyl, cyclobutyl, cyclopentyl, cyclohexyl, cycloheptyl, and cyclooctyl. The cycloalkyl group can be optionally substituted by one or more (e.g., 1 to 3) of the group independently selected from halogen, trifluoromethyl, trifluoromethoxy, hydroxyl, alkoxyl, nitro, cyano, alkylamino, and amino groups.

As used herein, the term “heterocycloalkyl” refers to a monocyclic or a bicyclic, saturated or partially unsaturated ring system having an indicated number (e.g., 4 to 12) of ring atoms, of which an indicated number (e.g., 1 to 5) of the ring atoms is(are) heteroatom independently selected from the group consisting of nitrogen, oxygen and sulfur, and the remaining ring atoms are carbon. Nonlimiting examples of heterocycloalkyl groups are azetidinyl, pyrrolidinyl, piperidinyl, piperazinyl, dihydropyrrolyl, morpholinyl, thiomorpholinyl, dihydropyridinyl, oxacycloheptyl, dioxacycloheptyl, thiacycloheptyl, diazacycloheptyl, each optionally substituted with one or more, and typically one to three, of the group independently selected from halogen, C₁₋₆ alkyl, C₁₋₆ alkoxy, cyano, amino, carbamoyl, nitro, carboxy, C₂₋₇ alkenyl, C₂₋₇ alkynyl, and the like on an atom of the ring. The heterocycloalkyl can link to other parts of the molecule via a carbon atom or a heteroatom. The heterocycloalkyl group can be substituted by one or more (e.g., 1 to 3) of the group independently selected from halogen, C₁₋₆ alkyl, C₁₋₆ alkoxy, cyano, amino, carbamoyl, nitro, carboxy, C₂₋₇ alkenyl, C₂₋₇ alkynyl, and the like on an atom of the ring.

As used herein, the term “heterocycle” refers to a 5-6 membered aromatic or nonaromatic ring containing 1 to 4 heteroatom(s) independently selected from the group consisting of nitrogen, oxygen and sulfur. “Heterocycle” includes the heteroaryl as mentioned above; it also includes dihydro- and tetrahydro-analogs of the heteroaryl. “Heterocycle” includes, but is not limit to, imidazolyl, indolyl, isothiazolyl, isoxazolyl, oxadiazolyl, oxazolyl, oxetanyl, pyranyl, pyrazinyl, pyrazolyl, pyridazinyl, pyridyl, pyrimidinyl, pyrrolyl, quinazolinyl, tetrazolyl, thiadiazolyl, thiazolyl, thienyl, triazolyl, 1,4-dioxanyl, pyrrolidinyl, dihydro-imidazolyl, dihydro-isoxazolyl, dihydro-isothiazolyl, dihydro-oxadiazolyl, dihydro-oxazolyl, dihydro-pyrazinyl, dihydro-pyrazolyl, dihydro-pyridyl, dihydro-pyrimidinyl, dihydro-pyrrolyl, dihydro-tetrazolyl, dihydro-thiadiazolyl, dihydro-thiazolyl, dihydro-thienyl, dihydro-triazolyl, methylene dioxy-benzophenone acyl, tetrahydrofuranyl, tetrahydrothiopheneyl, and their N-oxides etc. The linkage of heterocycle substituent can be achieved through carbon atom or heteroatom. In some embodiments, heterocycle is imidazolyl, pyridyl, 1-pyrrolidone, 2-piperidone, 2-pyrimidone, 2-pyrrolidone, thienyl, oxazolyl, triazolyl, isoxazolyl, etc.

Unless specially mentioned, alkyl, cycloalkyl, aryl, heteroaryl and heterocyclic groups can be substituted or not be substituted. For example, C₁-C₆ alkyl group can be substituted by one, two, or three substituents independently selected from the group consisting of OH, halogen, alkoxyl, dialkylamino, or heterocyclic ring such as morpholinyl, piperidinyl groups.

When any variable (such as R) occurs in the constitution or structure of the compound more than once, the definition of the variable at each occurrence is independent. Thus, for example, if a group is substituted with 0-2 R, the group can be optionally substituted with up to two R, wherein the definition of R at each occurrence is independent. Moreover, a combination of the substituent and/or the variant thereof is allowed only when the combination results in a stable compound.

When a group is indicated to be substituted but the number of the substituent is not provided, it means that the number of the substituent be one or more, e.g., 1, 2, 3 or 4.

In some embodiments, some variables in the formula I are as defined below.

In some embodiments,

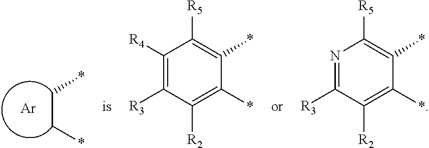

In some embodiments, ring B is

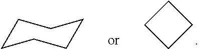

In some embodiments, R^{c} and R^{d} are F and F; H and H; OH and CH₃; CH₃ and CH₃; CH₃ and OH; H and OH; CH₂CH₃ and CH₂CH₃; or, CH₂OH and CH₂OH.

In some embodiments,

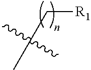

is H, CH₃, or CH₂CH₃.

In some embodiments, R₂ is H; R₃ is halogen; and, R₄ and R₅ are H.

In some embodiments, R₇ is halogen; each of R₈, R₉, and R₁₀ is H.

In some embodiments, R^{c} is —C(═O)OH, —C(═O)NH₂, or —C(═O)NHSO₂CH₃.

In some embodiments, the compound of formula (I) is selected from the group consisting of

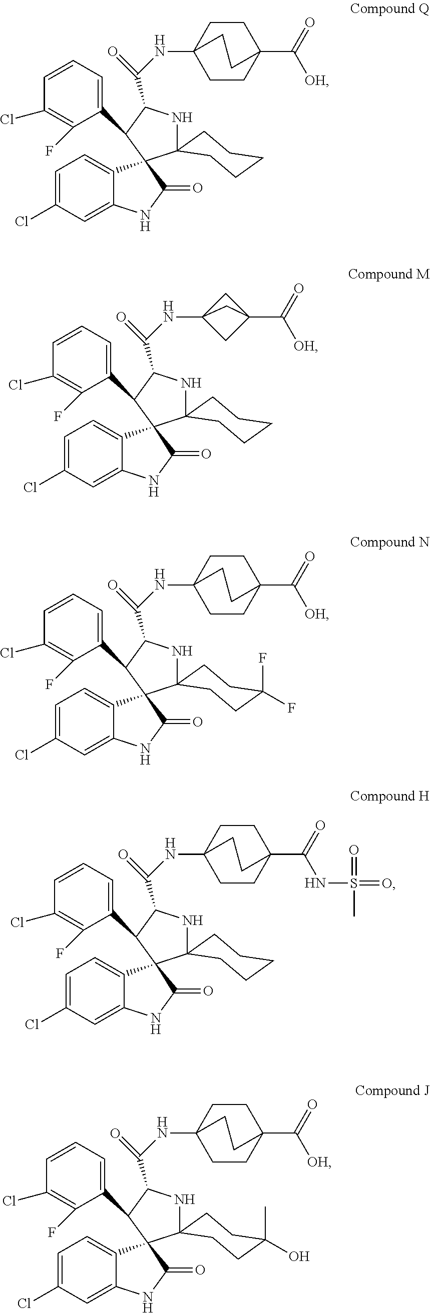 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CC(C(=O)O)(C1)C2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

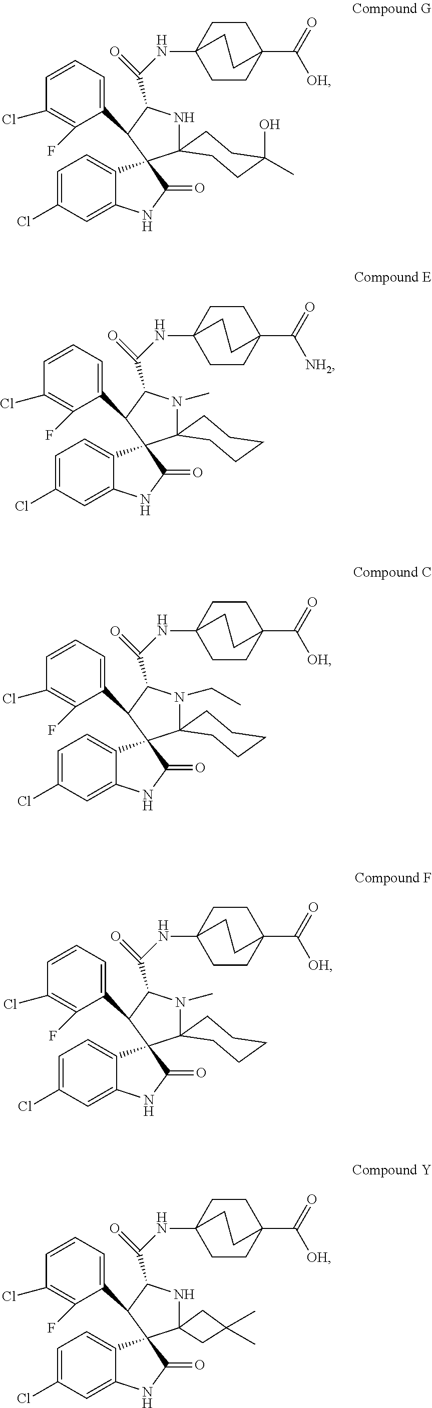 [SMILES: CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ccc21]

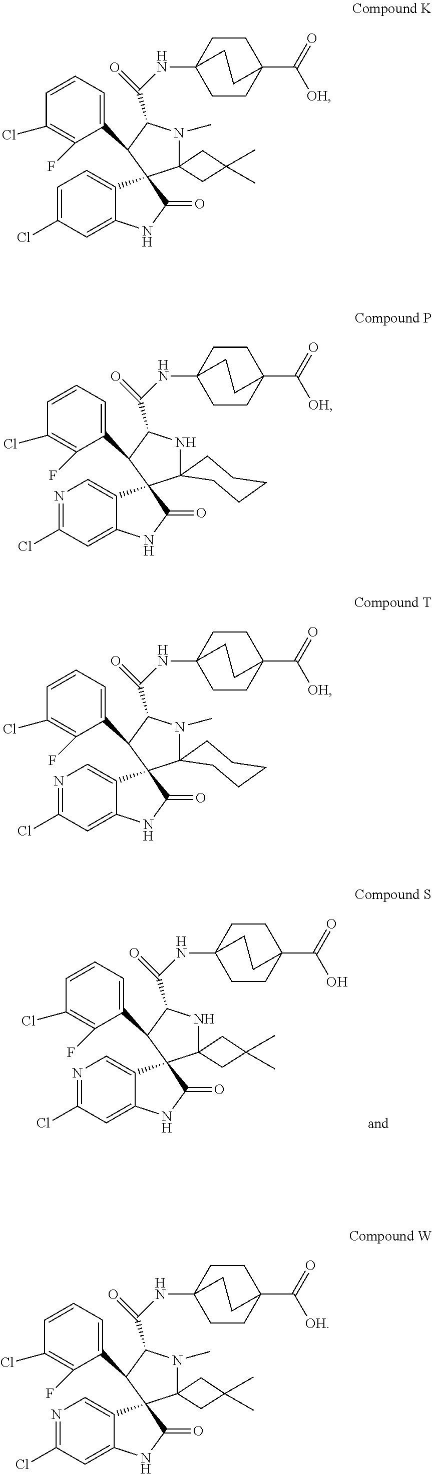 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CC(C)(C)C2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ncc21 ; CN1[C@@H](C(=O)NC23CCC(C(=O)O)(CC2)CC3)[C@H](c2cccc(Cl)c2F)[C@]2(C(=O)Nc3cc(Cl)ncc32)C12CC(C)(C)C2]

In some embodiments, the compound of formula I

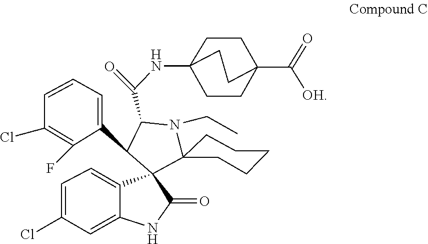 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

In some embodiments, the treatment is a monotherapy with the compound of formula I or the pharmaceutically acceptable salt thereof.

In some embodiments, the treatment is a combination therapy with the compound of formula I or the pharmaceutically acceptable salt thereof in combination with at least one additional anti-cancer agent. Accordingly, also provided herein is a combination of the compound of formula I or the pharmaceutically acceptable salt thereof and at least one additional anti-cancer agent for use in the treatment of the disease described above.

In some embodiments, the additional anti-cancer agent can be antimetabolite (e.g., 5-azacitidine or cytarabine).

In some embodiments, the additional anti-cancer agent can be anticancer reagents, wherein the anticancer reagents are preferably selected of immuno-modulatory drugs (e.g., pomalidomide, thalidomide or lenalidomide) and/or dexamethasone.

In some embodiments, the anticancer reagents are immuno-modulatory drugs, or the anticancer reagents are immuno-modulatory drugs and dexamethasone.

In some embodiments, the immuno-modulatory drug is pomalidomide or lenalidomide, preferably, pomalidomide.

The compound of formula I or the pharmaceutically acceptable salt thereof and the additional anti-cancer agent can be administered separately or simultaneously and can be administered through identical or different routes of administration. The separated administration may be close in time or distant in time but make sure the compound of formula I or the pharmaceutically acceptable salt thereof and the additional anti-cancer agent can act in concert so as to provide the desired therapeutic effect. For example, the compound of formula I or the pharmaceutically acceptable salt thereof and the additional anti-cancer agent can be administered sequentially at a time interval of about 1 minute to about 72 hours, e.g., about 1 minute, about 5 minutes, about 10 minutes, about 15 minutes, about 30 minutes, about 45 minutes, about 1 hour, about 2 hours, about 4 hours, about 6 hours, about 12 hours, about 24 hours.

In some embodiments, the treatment is a combination therapy with the compound of formula I or the pharmaceutically acceptable salt thereof in combination with 5-azacitidine.

In some embodiments, the treatment is a combination therapy with the compound of formula I or the pharmaceutically acceptable salt thereof in combination with cytarabine.

In some embodiments, the treatment is a combination therapy with the compound of formula I or the pharmaceutically acceptable salt thereof in combination with pomalidomide or lenalidomide, preferably pomalidomide.

In some embodiments, the treatment is a combination therapy with the compound of formula I or the pharmaceutically acceptable salt thereof in combination with pomalidomide and dexamethasone.

In some embodiments, the subject to be treated has AML-MRC accompanied with one or more mutations of FLT3 and IDH1/2.

In some embodiments, the subject to be treated has AML-MRC with no naive AML.

In some embodiments, the AML-MRC is relapsed or refractory AML-MRC.

In some embodiments, the AML-MRC is refractory or relapse AML-MRC after treatment with one or more therapeutical agents selected from the group of 5-azacitidine, venetoclax, decitabine, cytarabine, aclarubicin, and G-CSF. In some embodiments, the AML-MRC is refractory or relapse after treatment with venetoclax plus decitabine. In some embodiments, the AML-MRC is refractory or relapse after treatment with decitabine followed by cytarabine, aclarubicin, and G-CSF (i.e., D-CAG treatment).

In some embodiments, the subject to be treated has MDS with excess blasts.

In some embodiments, the MDS is relapsed or progressed MDS.

In some embodiments, the MDS is relapsed or progressed MDS after treatment with one or more therapeutical agents selected from the group of decitabine and lenalidomide.

In some embodiments, the subject to be treated is an adult.

Administration regimen of the compound of formula I or the pharmaceutically acceptable salt thereof

The disclosure described herein apply to both monotherapy and combination therapy with the compound of formula I (e.g., Compound C) or the pharmaceutically acceptable salt thereof.

In some embodiments, the compound of formula I or the pharmaceutically acceptable salt thereof is administered in an amount of 100 to 250 mg/day (e.g., 100, 150, 200, 250 mg/day).

In some embodiments, the compound of formula I or the pharmaceutically acceptable salt thereof is administered orally. Formulations suitable for oral administration are known in the art, including but not limited to solid, semi-solid and liquid systems such as tablets, capsules, liquids, and powders, which can be prepared by conventional methods in the art. In some embodiments, the compound of formula I or the pharmaceutically acceptable salt thereof is formulated as a capsule.

In some embodiments, the compound of formula I or the pharmaceutically acceptable salt thereof is administered orally every day.

In some embodiments, the compound of formula I or the pharmaceutically acceptable salt thereof is administered orally every day for consecutive 5-7 days.

In some embodiments, the compound of formula I or the pharmaceutically acceptable salt thereof is administered orally in an amount of 100 to 250 mg/day, e.g., 100, 150, 200, 250 mg/day, for e.g., consecutive 5-7 days (e.g., consecutive 5 days).

In some embodiments, the compound of formula I or the pharmaceutically acceptable salt thereof is administered once a day.

In some embodiments, the treatment comprises at least one 28-day treatment cycle, wherein the compound of formula I or the pharmaceutically acceptable salt thereof is administered on day 1 to 5 of each treatment cycle. In some embodiments, the treatment comprises at least one 28-day treatment cycle, wherein the compound of formula I or the pharmaceutically acceptable salt thereof is administered orally every day in an amount of 100 to 250 mg/day (e.g., 100, 150, 200, 250 mg/day) on day 1 to 5 of each treatment cycle.

In some embodiments, the treatment comprises at least one 28-day treatment cycle, wherein the compound of formula I or the pharmaceutically acceptable salt thereof is administered on day 1 to 7 of each treatment cycle. In some embodiments, the treatment comprises at least one 28-day treatment cycle, wherein the compound of formula I or the pharmaceutically acceptable salt thereof is administered orally every day in an amount of 100 to 250 mg/day (e.g., 100, 150, 200, 250 mg/day) on day 1 to 7 of each treatment cycle.

In some embodiments, the treatment comprises administering to the subject (i) the compound of formula I or the pharmaceutically acceptable salt thereof; in combination with (ii) 5-azacitidine.

In some embodiments, the 5-azacitidine is subcutaneously (SC) or intravenously (IV) injected.

In some embodiments, the 5-azacitidine is subcutaneously or intravenously injected every day.

In some embodiments, the 5-azacitidine is subcutaneously or intravenously injected every day for consecutive 7 days.

In some embodiments, the 5-azacitidine is administered in an amount of 75 mg/m²/day. In some embodiments, the 5-azacitidine is subcutaneously or intravenously injected in an amount of 75 mg/m²/day.

In some embodiments, the 5-azacitidine is administered once a day.

In some embodiments, the treatment comprises at least one 28-day treatment cycle, wherein the compound of formula I or the pharmaceutically acceptable salt thereof is administered on day 1 to 5 of each treatment cycle, and the 5-azacitidine is administered on 7 out of 9 days from day 1 to day 9 (e.g., on day 1 to 7) of each treatment cycle. In some embodiments, the treatment comprises at least one 28-day treatment cycle, wherein the compound of formula I or the pharmaceutically acceptable salt thereof is administered orally every day in an amount of 100 to 250 mg/day (e.g., 100, 150, 200, 250 mg/day) on day 1 to 5 of each treatment cycle, and the 5-azacitidine is subcutaneously or intravenously injected in an amount of 75 mg/m²/day every day on 7 out of 9 days from day 1 to day 9 (e.g., on day 1 to 7) of each treatment cycle.

In some embodiments, the treatment comprises at least one 28-day treatment cycle, wherein the compound of formula I or the pharmaceutically acceptable salt thereof is administered on day 1 to 7 of each treatment cycle, and the 5-azacitidine is administered on day 1 to 7 of each treatment cycle. In some embodiments, the treatment comprises at least one 28-day treatment cycle, wherein the compound of formula I or the pharmaceutically acceptable salt thereof is administered orally every day in an amount of 100 to 250 mg/day (e.g., 100, 150, 200, 250 mg/day) on day 1 to 7 of each treatment cycle, and the 5-azacitidine is subcutaneously injected in an amount of 75 mg/m²/day every day on day 1 to 7 of each treatment cycle.

In some embodiments, the treatment comprises administering to the subject (i) the compound of formula I or the pharmaceutically acceptable salt thereof; in combination with (ii) cytarabine.

In some embodiments, the cytarabine is intravenously administered.

In some embodiments, the cytarabine is intravenously administered every day.

In some embodiments, the cytarabine is intravenously administered every day for consecutive 5 days.

In some embodiments, the cytarabine is administered in an amount of 1 g/m²/day. In some embodiments, the cytarabine is intravenously administered in an amount of 1 g/m²/day.

In some embodiments, the cytarabine is administered once a day.

In some embodiments, the treatment comprises administering to the subject (i) the compound of formula I or the pharmaceutically acceptable salt thereof; in combination with (ii) pomalidomide.

In some embodiments, the treatment comprises administering to the subject (i) the compound of formula I or the pharmaceutically acceptable salt thereof; in combination with (ii) lenalidomide.

In some embodiments, the treatment comprises administering to the subject (i) the compound of formula I or the pharmaceutically acceptable salt thereof; in combination with (ii) pomalidomide and (iii) dexamethasone.

In some embodiments, pomalidomide is oral administration (PO).

In some embodiments, lenalidomide is oral administration (PO).

In some embodiments, pomalidomide or lenalidomide is oral administration

In some embodiments, the immuno-modulatory drugs (e.g., pomalidomide, thalidomide or lenalidomide) is administered in an amount of 0.1 to 1.0 mg/kg/day (e.g., 0.1, 0.25, 0.50, 0.75, 1.0 mg/day).

In some embodiments, the immuno-modulatory drugs (e.g., pomalidomide, thalidomide or lenalidomide) is administered orally every day.

In some embodiments, the immuno-modulatory drugs (e.g., pomalidomide, thalidomide or lenalidomide) is administered orally every day for consecutive 5-7 days.

In some embodiments, the immuno-modulatory drugs (e.g., pomalidomide, thalidomide or lenalidomide) is administered once a day.

In some embodiments, dexamethasone is subcutaneously (SC) or intravenously (IV) injected.

In some embodiments, dexamethasone is subcutaneously or intravenously injected twice a week.

In some embodiments, dexamethasone is subcutaneously or intravenously injected twice a week for consecutive 7 days.

In some embodiments, dexamethasone is administered in an amount of 3 mg/kg. In some embodiments, the dexamethasone is subcutaneously or intravenously injected in an amount of 3 mg/kg.

In some embodiments, the treatment comprises at least one 28-day treatment cycle, wherein the compound of formula I or the pharmaceutically acceptable salt thereof is administered on day 1 to 7 of each treatment cycle, and the cytarabine is administered on day 3 to 7 of each treatment cycle. In some embodiments, the treatment comprises at least one 28-day treatment cycle, wherein the compound of formula I or the pharmaceutically acceptable salt thereof is administered orally every day in an amount of 100 to 250 mg/day (e.g., 100, 150, 200, 250 mg/day) on day 1 to 7 of each treatment cycle, and the cytarabine is intravenously administered every day in an amount of 1 g/m²/day on day 3 to 7 of each treatment cycle.

In some embodiments, the above 28-day treatment cycle is repeated for 1, 2, 3, 4, 5, or 6 times, or until a clinical benefit (e.g., complete remission, complete remission with incomplete hematologic recovery) is observed.

In some embodiments, the treatment comprises at least one 21-day to 28-day treatment cycle, wherein, the compound of formula I or the pharmaceutically acceptable salt thereof (e.g., compound C, that is Alrizomadlin) is administered orally in an amount of 100 to 250 mg/day (e.g., 100, 150, 200, 250 mg/day); the immuno-modulatory drug (e.g., pomalidomide, thalidomide or lenalidomide) is administered orally in an amount of 0.1 to 1.0 mg/kg/day; or the compound of formula I or the pharmaceutically acceptable salt thereof (e.g., compound C, that is Alrizomadlin) is administered orally in an amount of 100 to 250 mg/day (e.g., 100, 150, 200, 250 mg/day); the immuno-modulatory drug (e.g., pomalidomide, thalidomide or lenalidomide) is administered orally in an amount of 0.1 to 1.0 mg/kg/day, and the dexamethasone is subcutaneously or intravenously injected in an amount of 3 mg/kg.

In some embodiments, wherein, the treatment comprises at least one 21-day to 28-day treatment cycle, the compound of formula I or the pharmaceutically acceptable salt thereof (e.g., compound C, that is Alrizomadlin) is administered orally in an amount of 100 to 250 mg/day (e.g., 100, 150, 200, 250 mg/day), QD; the immuno-modulatory drug (e.g., pomalidomide, thalidomide or lenalidomide) is administered orally in an amount of 1 to 2.5 mg/day (e.g., 1, 1.5, 2.0, 2.5 mg/day), QD; or the compound of formula I or the pharmaceutically acceptable salt thereof (e.g., compound C, that is Alrizomadlin) is administered orally in an amount of 100 to 250 mg/day (e.g., 100, 150, 200, 250 mg/day), QD; the immuno-modulatory drug (e.g., pomalidomide, thalidomide or lenalidomide) is administered orally in an amount of 1 to 2.5 mg/day (e.g., 1, 1.5, 2.0, 2.5 mg/day), QD, and the dexamethasone is subcutaneously or intravenously injected in an amount of 6 to 15 mg/day (e.g., 6, 9, 12, 15 mg/day), BIW.

In some embodiments, the treatment comprises at least one 21-day to 28-day treatment cycle, wherein, the Alrizomadlin is administered orally in an amount of 4.00-4.10 mg/kg/day, QD, the pomalidomide is administered orally in an amount of 0.04-0.05 mg/kg/day, QD, or the Alrizomadlin is administered orally in an amount of 4.00-4.10 mg/kg/day, QD, the pomalidomide is administered orally in an amount of 0.04-0.05 mg/kg/day, QD, and the dexamethasone is subcutaneously or intravenously injected in an amount of 0.24-0.25 mg/kg/day, BIW.

In some embodiments, the present disclosure provides a pharmaceutical combination, wherein, comprising (i) the compound of formula I or the pharmaceutically acceptable salt thereof and (ii) one or more anticancer reagents, wherein the anticancer reagents are selected of antimetabolite (e.g., 5-azacitidine or cytarabine), immuno-modulatory drugs (e.g., pomalidomide, thalidomide or lenalidomide) and/or dexamethasone.

In some embodiments, the present disclosure provides a pharmaceutical combination, wherein, comprising (i) the compound of formula I or the pharmaceutically acceptable salt thereof (e.g., compound C, that is Alrizomadlin) and (ii) 5-azacitidine or cytarabine; wherein, the compound of formula I or the pharmaceutically acceptable salt thereof is administered orally in an amount of 100 to 250 mg/day (e.g., 100, 150, 200, 250 mg/day), QD; and (ii) 5-azacitidine is subcutaneously injected in an amount of 75 mg/m²/day every day or the cytarabine is intravenously administered in an amount of 1 g/m²/day.

In some embodiments, the present disclosure provides a pharmaceutical combination, wherein, comprising (i) the compound of formula I or the pharmaceutically acceptable salt thereof (e.g., compound C, that is Alrizomadlin) and (ii) the immuno-modulatory drugs (e.g., pomalidomide, thalidomide or lenalidomide), or the immuno-modulatory drugs (e.g., pomalidomide, thalidomide or lenalidomide) and the dexamethasone; wherein, the compound of formula I or the pharmaceutically acceptable salt thereof is administered orally in an amount of 100 to 250 mg/day (e.g., 100, 150, 200, 250 mg/day), QD; and (ii) the immuno-modulatory drugs (e.g., pomalidomide, thalidomide or lenalidomide) is subcutaneously injected in an amount of 0.1 to 1.0 mg/kg/day (e.g., 0.1, 0.25, 0.50, 0.75, 1.0 mg/day); or the compound of formula I or the pharmaceutically acceptable salt thereof is administered orally in an amount of 100 to 250 mg/day (e.g., 100, 150, 200, 250 mg/day), QD; and (ii) the immuno-modulatory drugs (e.g., pomalidomide, thalidomide or lenalidomide) is subcutaneously injected in an amount of 0.1 to 1.0 mg/kg/day (e.g., 0.1, 0.25, 0.50, 0.75, 1.0 mg/day), and the dexamethasone is subcutaneously or intravenously injected in an amount of 3 mg/kg.

In some embodiments, the present disclosure provides a pharmaceutical combination, wherein, comprising (i) the compound of formula I or the pharmaceutically acceptable salt thereof (e.g., compound C, that is Alrizomadlin) and (ii) the immuno-modulatory drugs (e.g., pomalidomide, thalidomide or lenalidomide), or the immuno-modulatory drugs (e.g., pomalidomide, thalidomide or lenalidomide) and the dexamethasone; wherein, the compound of formula I or the pharmaceutically acceptable salt thereof is administered orally in an amount of 100 to 250 mg/day (e.g., 100, 150, 200, 250 mg/day), QD; and (ii) the immuno-modulatory drugs (e.g., pomalidomide, thalidomide or lenalidomide) is subcutaneously injected in an amount of 1 to 2.5 mg/day (e.g., 1, 1.5, 2.0, 2.5 mg/day), QD; or the compound of formula I or the pharmaceutically acceptable salt thereof is administered orally in an amount of 100 to 250 mg/day (e.g., 100, 150, 200, 250 mg/day), QD; and (ii) the immuno-modulatory drugs (e.g., pomalidomide, thalidomide or lenalidomide) is subcutaneously injected in an amount of 1 to 2.5 mg/day (e.g., 1, 1.5, 2.0, 2.5 mg/day), QD, and the dexamethasone is subcutaneously or intravenously injected in an amount of 6 to 15 mg/day (e.g., 6, 9, 12, 15 mg/day), BIW.

In some embodiments, the present disclosure provides a pharmaceutical combination, wherein, comprising (i) the compound of formula I or the pharmaceutically acceptable salt thereof (e.g., compound C, that is Alrizomadlin) and (ii) the immuno-modulatory drugs (e.g., pomalidomide, thalidomide or lenalidomide), or the immuno-modulatory drugs (e.g., pomalidomide, thalidomide or lenalidomide) and the dexamethasone; wherein, the compound of formula I or the pharmaceutically acceptable salt thereof is administered orally in an amount of 4.00-4.10 mg/kg/day, QD; and (ii) the immuno-modulatory drugs (e.g., pomalidomide, thalidomide or lenalidomide) is subcutaneously injected in an amount of 0.04-0.05 mg/kg/day, QD; or the compound of formula I or the pharmaceutically acceptable salt thereof is administered orally in an amount of 4.00-4.10 mg/kg/day, QD; and (ii) the immuno-modulatory drugs (e.g., pomalidomide, thalidomide or lenalidomide) is subcutaneously injected in an amount of 0.04-0.05 mg/kg/day, and the dexamethasone is subcutaneously or intravenously injected in an amount of 0.24-0.25 mg/kg/day, BIW.

In some embodiments, the present disclosure provides a pharmaceutical composition, comprising (i) the compound of formula I or a pharmaceutically acceptable salt thereof and (ii) immuno-modulatory drugs (e.g., pomalidomide, thalidomide or lenalidomide), wherein, the mass ratio of (i) the compound of formula I or a pharmaceutically acceptable salt thereof/(ii) immuno-modulatory drugs is 100:1-1:100; for example 100:1, 50:1, 20:1, 10:1, 5:1, 1:1, 1:5, 1:10, 1:20, 1:30.

In some embodiments, the present disclosure provides a pharmaceutical composition, comprising (i) the compound of formula I or a pharmaceutically acceptable salt thereof and (ii) immuno-modulatory drugs (e.g., pomalidomide, thalidomide or lenalidomide), wherein, the mass ratio of (i) the compound of formula I or a pharmaceutically acceptable salt thereof/(ii) immuno-modulatory drugs is 100:1-1:100 (e.g., 100:1, 50:1, 20:1, 10:1, 5:1, 1:1, 1:5, 1:10, 1:20, 1:30); such as comprising (i) the amount of the compound of formula I or the pharmaceutically acceptable salt thereof is 100 to 250 mg (e.g., 100, 150, 200, 250 mg) and (ii) the amount of the immuno-modulatory drugs (e.g., pomalidomide, thalidomide or lenalidomide) is 1 to 2.5 mg (e.g., 1, 1.5, 2.0, 2.5 mg).

The reagents used in the present disclosure are commercially available.

The compound of formula I and the synthesis thereof have been disclosed in WO2015161032A1, the contents of which are incorporated herein by reference in their entireties.

Without violating the common sense in the art, the above preferred conditions can be arbitrarily combined, then preferred embodiments of the invention are obtained.

### EXAMPLES

The following examples further illustrate the present disclosure, but the present disclosure is not limited thereto.

In the below examples, Compound C was obtained by the methods disclosed in WO2015161032A1, and was prepared into capsules.

### Example 1: A Phase Ib/II Study of Compound C Alone or in Combination with Azacitidine in Patients with Relapse/Refractory AML, CMML or MDS

This was a two part study in patients with relapsed/refractory (R/R) acute myeloid leukemia (AML), Chronic Myelomonocytic Leukemia (CMML) and relapsed/progressed higher-risk myelodysplastic syndrome (MDS), that was designed to evaluate the safety and tolerability of Compound C as a single agent in Part I, followed by a combination of Compound C plus 5-azacitidine in Part II.

Part I: Dose escalation of Compound C will use standard 3+3 design. Compound C will be administered orally once daily (QD) on Day 1 to 5 every 28-day cycle. The starting target dose is 100 mg (dose level; DL1) and will be increased in subsequent cohorts to 150 mg (DL2), 200 mg (DL3) and 250 mg (DL4), accordingly.

Part II: Compound C will be co-administered with 5-azacitidine SC or IV at 75 mg/m² once daily on 7 out of 9 days from Day 1 to Day 9 every 28-day cycle.

### Inclusion Criteria

1. Patients with a diagnosis of histologically confirmed relapsed or refractory (R/R) acute myeloid leukemia (AML), R/R Chronic Myelomonocytic Leukemia (CMML) or relapsed/refractory Higher-Risk MDS (Overall Revised International Prognostic Scoring System (IPSS—R) score >3, including intermediate, high or very high risk) by WHO classification for which no available standard therapies are indicated or anticipated to result in a durable response.

2. Age >/=18 years.

3. Adequate organ function

4. ECOG performance status of <2.

5. A negative serum pregnancy test is required within 1 week for all women of childbearing potential prior to enrolling on this trial.

6. Patient must have the ability to understand the requirements of the study and signed informed consent.

7. Subject must have a projected life expectancy of at least 12 weeks.

8. Subject has a white blood cell count<25×10⁹/L.

### Exclusion Criteria:

1. Uncontrolled intercurrent illness including, but not limited to active uncontrolled infection, symptomatic congestive heart failure (NYHA Class III or IV), unstable angina pectoris, clinically significant cardiac arrhythmia, or psychiatric illness/social situations that would limit compliance with study requirements.

2. Have had leukemia therapy for 14 days prior to starting investigational drug.

3. Have acute promyelocytic leukemia.

4. Active infection requiring systemic antibiotic/antifungal medication, known clinically active hepatitis B or C, or HIV infection.

5. Documented hypersensitivity to any of the components of the therapy program.

6. Active, uncontrolled CNS leukemia will not be eligible.

7. Pregnant women.

8. Men and women of childbearing potential who do not practice contraception.

9. Any prior systemic MDM2-p53 inhibitor treatment

10. History of other malignancies within 2 years prior to study entry.

11. Failure to have recovered from prior treatment.

12. Significant screening electrocardiogram (ECG) abnormalities.

Results are shown in table 1:

TABLE 1

| | | | | | End of cycle 1 |
|---|---|---|---|---|---|
| | Dosage | | | Prior | disease |
| Part | regimen | Subject | Diagnosis | treatment | assessment |
| Part I | Compound | Male | AML-MRC | venetoclax + | CRi |
| | C alone, | aged | (with TP53w, | decitabine | (Blast reduced |
| | 150 mg | 76 | FLT3-, | | from 43% |
| | | | IDH1/2-) | | to 13% |
| | | | | | C2D1, to 3% |
| | | | | | C3D1) |

Note:
CRi means complete remission with incomplete hematologic recovery. C2D1 means Day 1 of cycle 2; C3D1 means Day 1 of cycle 3.

### Example 2: A Phase Ib/II Study of Compound C Alone or in Combination with 5-Azacitidine or Cytarabine

This was a three parts study in patients with relapsed/refractory acute myeloid leukemia (AML), relapsed or progressed high or very high risk myelodysplastic syndrome (MDS) that was designed to evaluate the safety and tolerability of Compound C as a single agent in Part I, followed by a combination dose escalation of Compound C plus either 5-azacitidine or cytarabine in Part II and a dose expansion of the combination treatment in part III.

Part I: Dose escalation of Compound C will use standard 3+3 design. Compound C will be administered orally once daily (QD) on Day 1 to 7 every 28-day cycle. The starting target dose is 100 mg (dose level; DL1) and will be increased in subsequent cohorts to 150 mg (DL2), 200 mg (DL3) and 250 mg (DL4), accordingly.

Part II: Dose escalation of Compound C combination with either 5-azacitidine or cytarabine, which includes two Arms, Arm A and Arm B. Arm A will enroll patients with relapsed or progressed high or very high risk MDS and Arm B will enroll patients with R/R AML. In this part, 5-azacitidine and cytarabine will be administered with fixed dose. The starting dose of Compound C was 100 mg and will be increased subsequently to 150 mg and 200 mg. In Arm A, Compound C will be co-administered with 5-azacitidine subcutaneously injected at 75 mg/m² once daily on Day 1 to 7 every 28-day cycle. In Arm B, Compound C will be co-administered once daily on Day 1 to 7 every 28-day cycle with cytarabine intravenously administered at 1 g/m² once daily on Day 3 to 7 every 28-day cycle.

Part III: This is the dose expansion part of Compound C combination with either 5-azacitidine or cytarabine at RP2D level of two Arms in part II. In this part, Compound C, 5-azacitidine and cytarabine will be administered at RP2D level as part III

### Inclusion Criteria

1. Patients with a diagnosis of histologically confirmed relapsed or refractory (R/R) acute myeloid leukemia by WHO classification or relapsed/progressed high/very high risk MDS (score>4.5) according to IPSS—R risk stratification.

2. Age >/=18 years.

3. Adequate organ function:

4. Subject must have a projected life expectancy of at least 12 weeks.

5. ECOG performance status of 0-1.

6. Patient must have the ability to understand the requirements of the study and signed informed consent. A signed informed consent by the patient or his legally authorized representative is required prior to their enrollment on the protocol.

7. Subject has a white blood cell count<50×10⁹/L.

### Exclusion Criteria:

1. Subject has acute promyelocytic leukemia.

2. Patients must not have had leukemia biotherapy 12 weeks prior to starting investigational drug, or less than 5 half-lives small molecular targeted drug therapy, or 28 days any anti-cancer therapy (whichever is longer).

3. Uncontrolled intercurrent illness including, but not limited to active uncontrolled infection, symptomatic congestive heart failure (NYHA Class III or IV), unstable angina pectoris, clinically significant cardiac arrhythmia, or psychiatric illness/social situations that would limit compliance with study requirements.

4. Active infection requiring systemic antibiotic/antifungal medication, known clinically active hepatitis B or C, or HIV infection.

5. Participants who have received allogeneic HSCT, or autologous HSCT within 12 months.

6. Patients with active, uncontrolled central nervous system (CNS) leukemia will not be eligible.

7. Any prior systemic MDM2-p53 inhibitor treatment

8. Any other condition or circumstance that would, in the opinion of the investigator, make the patient unsuitable for participation in the study.

9. Subject has a history of other malignancies within 5 years prior to study entry, with the exception of:
adequately treated in situ carcinoma of the cervix uteri or carcinoma in situ of breast;
basal cell carcinoma of the skin or localized squamous cell carcinoma of the skin;
previous malignancy confined and surgically resected (or treated with other modalities) with curative intention: requires discussion with sponsor.

TABLE 2

| | Dosage | | | Medical | |
|---|---|---|---|---|---|
| Part | regimen | Subject | Diagnosis | history | Response |
| Part I | Compound C | Male | R/R AML with | D-CAG | CRi |
| | alone, | aged 63 | myelodysplasia- | | (C1D62) |
| | 100 mg | | related changes | | |
| | Compound C | Male | R/R AML with | D-CAG | CRi |
| | alone, | aged 65 | myelodysplasia- | | (C3D21) |
| | 200 mg | | related changes | | |
| | Compound C | Male | R/R AML with | Decitabine + | MLFS |
| | alone, | aged 65 | myelodysplasia- | venetoclax, | |
| | 250 mg | | related changes | Azacitidine + | |
| | | | | venetoclax, | |
| | | | | D-CAG, | |
| | | | | Decitabine + | |
| | | | | Etoposide + | |
| | | | | Cytarabine + | |
| | | | | Doxorubicin | |

Note:
D-CAG means decitabine followed by cytarabine, aclarubicin, and G-CSF; CRi means complete remission with incomplete hematologic recovery; mCR means marrow complete remission. MLFS means Morphological non leukemic state. C1D62 means day 62 of cycle 1; C2D1 means Day 2 of cycle 1, etc. As table 2 shows, 3 R/R AML-MRC subjects were enrolled and received at least on cycle of Compound C monotherapy at 100 mg, 200 mg and 250 mg dose level, all of them were MDS related changes. All the subjects achieved response which includes 2 CRi and 1 MLFS.

### Example 3: In Vitro Cell Antiproliferation Assay

The anti-proliferative effect was determined by a water-soluble tetrazolium (WST)-based assay using Cell Counting Kit-8 (CCK-8). Cells were seeded in 96-well plates and treated with 9 serial concentrations of each test articles as single agent group, and one-to-one correspondence of 9 serial concentrations of two kinds of test articles were added into the same plate as combined group, respectively. Each concentration was tested in duplicates. Diluent were added into the same plate as cell control and blank control. The plate was then cultured at 37° C. in an incubator in an atmosphere with 5% CO₂ for 72 hours. At the end of treatments, CCK-8 reagent were directly added to the well that needs to be tested. The plate was incubated for 2-4 hours. The Optical density (OD) value was then detected at 450 nm on a microplate reader. The cells viability of single agent group was calculated using the mean OD value of duplicated wells following the equation below: Cell viability (%)=(OD sample−OD blank)/(OD cell control−OD blank)×100. The IC₅₀ was calculated with Graphpad Prism 9.1.0 Software using nonlinear regression type data analysis. For combined group, the combination index (CI) was calculated with Calcusyn 2.11 software, CI>1 indicated antagonism, CI=1 indicated additive, CI<1 indicated synergism.

### Detection of Cell Apoptosis and Cycle Arrest

Apoptotic cells were determined by using an Annexin V—PI (propidium iodide) staining kit. Briefly, Cells were harvested after treatments with single or combined test articles for 24 hours, then washed with ice-cold phosphate buffered saline (PBS). Cells were then stained with Annexin-V and Propidium (PI) according to the instructions of AnnexinV-FITC apoptosis detection Kit (Biyuntian, #1062L), and incubated for 10-20 minutes protecting from light at room temperature. The apoptotic cells were analyzed with a flow cytometer.

Cells were harvested after treatments with single or combined test articles for 24 hours, then washed with ice-cold phosphate buffered saline (PBS). Cells were resuspended with 70% ethanol and fixed overnight at 4° C. according to the instructions of the cell cycle and Apoptosis detection kit, then washed with PBS, after stained with PI, the cells were incubated at 37° C. for 30 minutes protecting from light. The cell cycle arrest were analyzed with a flow cytometer.

### Western Blotting Assay

After treatments with single or combined test articles, cultured cells were harvested and washed with ice-cold PBS. Cell pellets were lysed in RIPA buffer containing 1% PMSF, 1% phosphatase inhibitor, and 1% protease inhibitors. Protein concentrations were determined using the BCA Protein Assay Kit. Whole tumor lysates (20-50 g) were separated on an 8-12% SDS-PAGE. The separated proteins were transferred to a PVDF membrane. The PVDF membrane was blocked with 1-3% BSA buffer for 30 min to 1 hour at room temperature. Incubate membrane with diluted primary antibody in 1×TBST containing 1-3% BSA at 4° C., with gentle agitation overnight. Wash the membrane with 1×TBST three times. Incubate membrane with HRP-conjugated secondary antibody with gentle agitation for 1 hour at room temperature. Wash the membrane with 1×TBST three times. The signals were visualized with Super ECL Detection Reagent and detected by chemiluminescence imaging system.

### Antitumor Activity in Human MM Cells Derived Xenografts Model in Mice

Female nude mice, 4-6 weeks old, 16-18+20% g, were provided by Gempharmatech Co., Ltd. NCI-H929 cells were subcutaneously implanted at concentration of 5×10⁶ cells/animal to the mice at right flank. When tumor volume reached 100-200 mm³, animals were randomized to different group and started treatment. Body weight and tumor volume were recorded twice per week. Animal studies were conducted in SPF lab of Animal Science Department of Shanghai Family Planning Research Institute (Shanghai, China). All protocols of animal studies were approved by Institutional Animal Care and Use Committee (IACUC) of Animal Science Department of Shanghai Family Planning Research Institute (Shanghai, China). Synergistic effect was analysis using following equation: synergy score=((A/C)×(B/C))/(AB/C); A=RTV for compound A; B=B RTV for compound B; C=RTV for vehicle control, AB=RTV for combination of A and B (Clarke R. Issues in experimental design and endpoint analysis in the study of experimental cytotoxic agents in vivo in breast cancer and other models[J]. Breast Cancer Research & Treatment, 1997, 46(2-3):255-278). Synergy score>1, synergism; =1, additive; <1, antagonism.

### Result

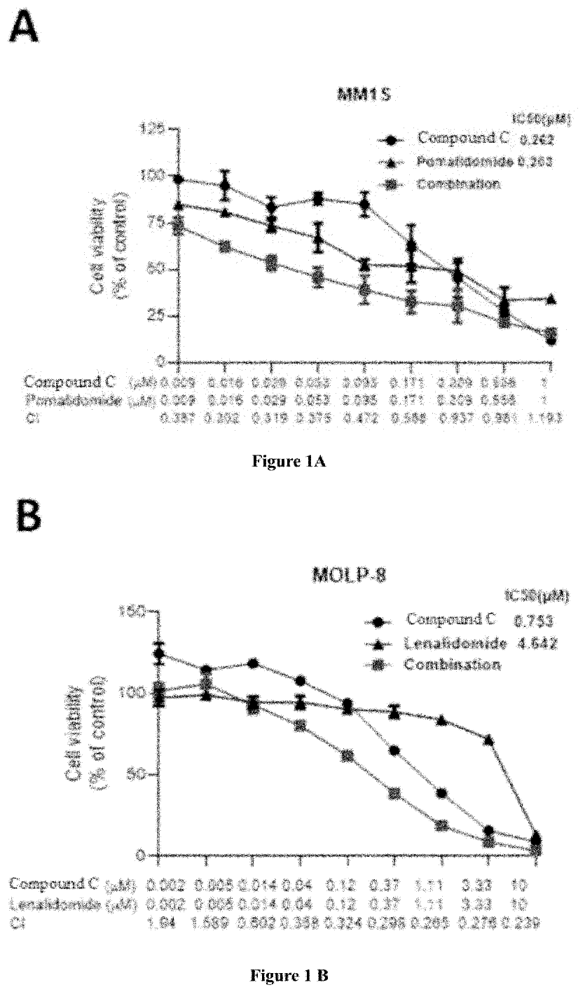

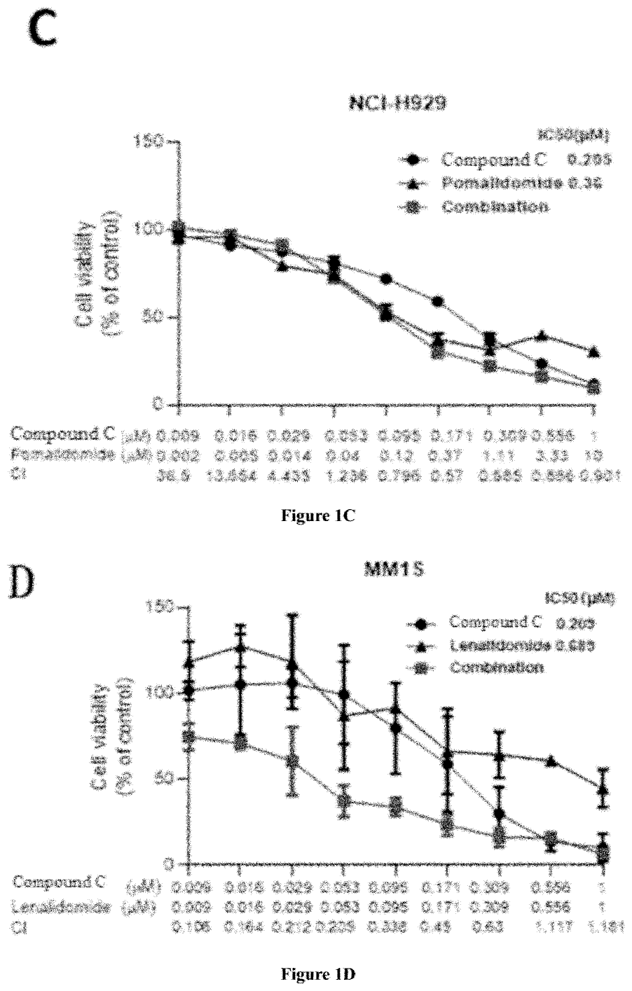

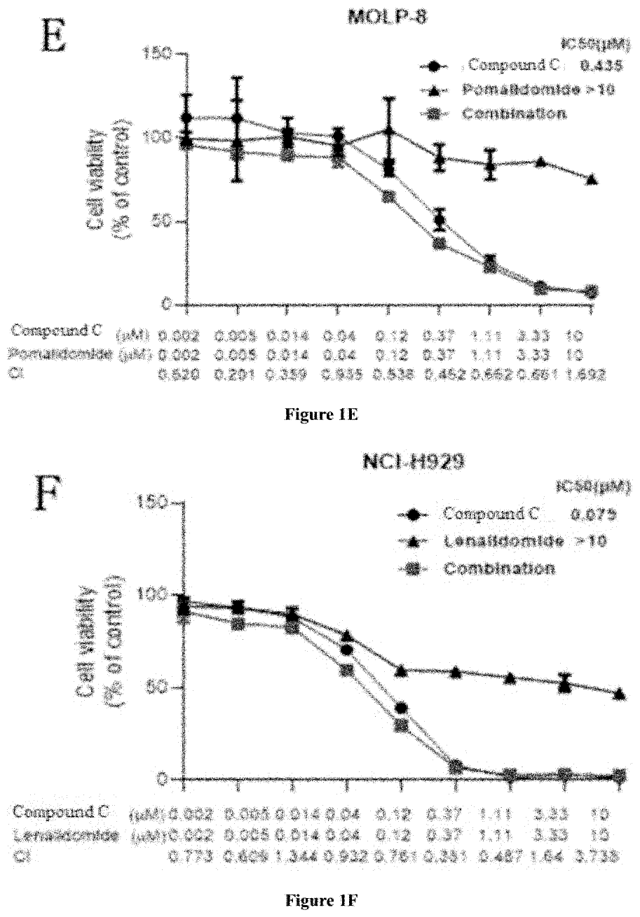

The cell-based antiproliferation studies suggested synergistic activity between Compound C and IMiDs against wild-type TP53 (TP53WT) MM cell lines, including MOLP-8, H929, and MM1S. FIG. 1 shows the synergistic anti-proliferation effects of Compound C and IMiDs (pomalidomide and lenalidomide) in MM MM1S (A) MOLP-8 (B) and H929 (C) cell lines. Combination index (CI) was calculated using CalcuSyn (Biosoft) software. A CI value <1.0 indicates synergism.

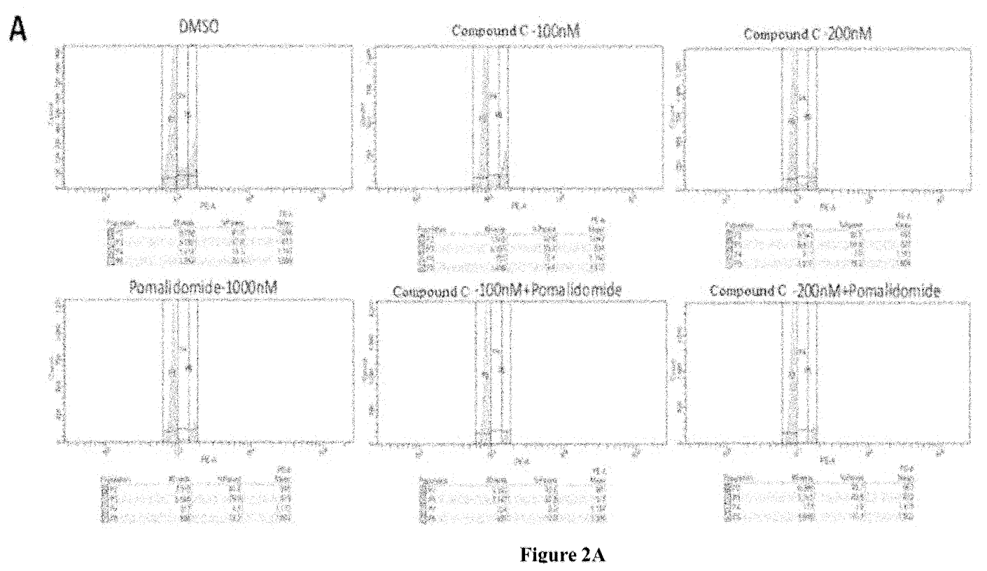

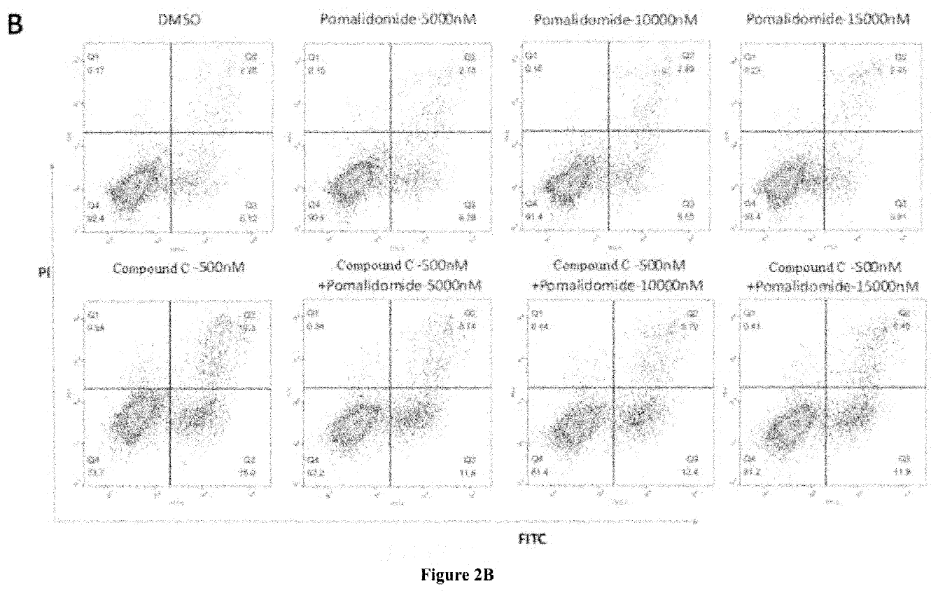

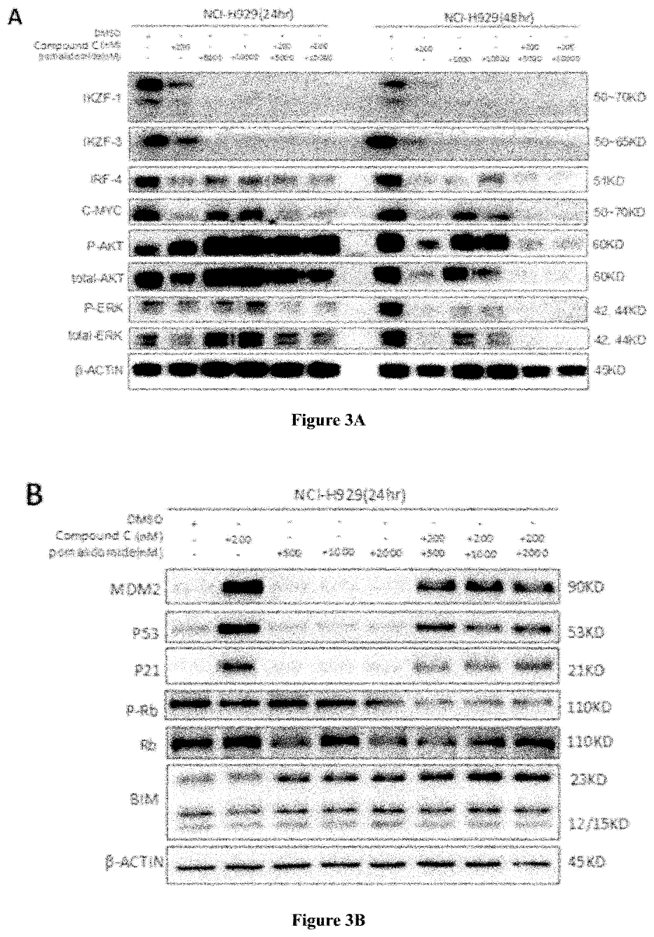

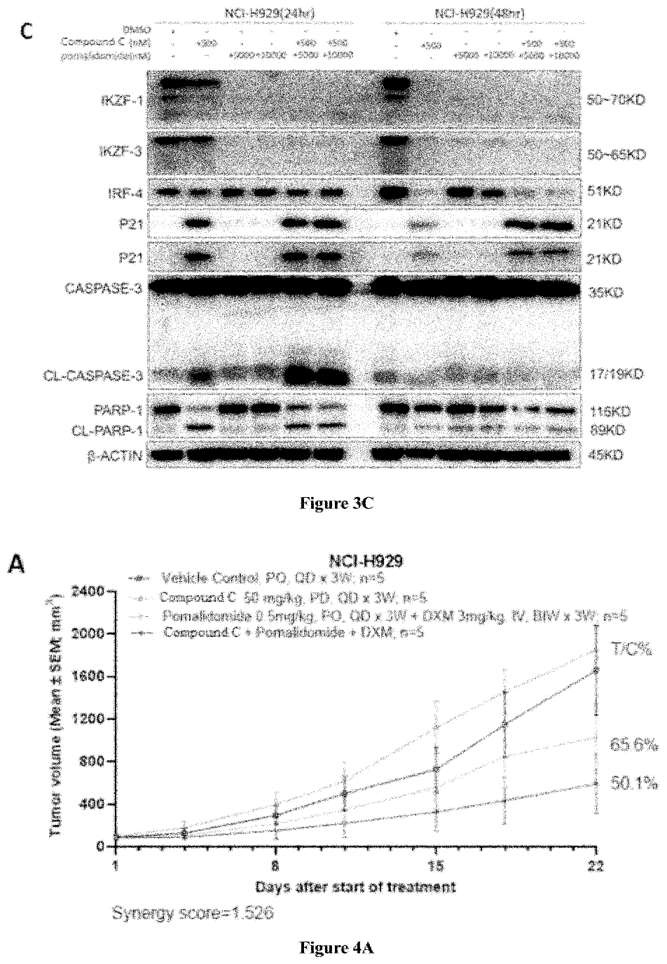

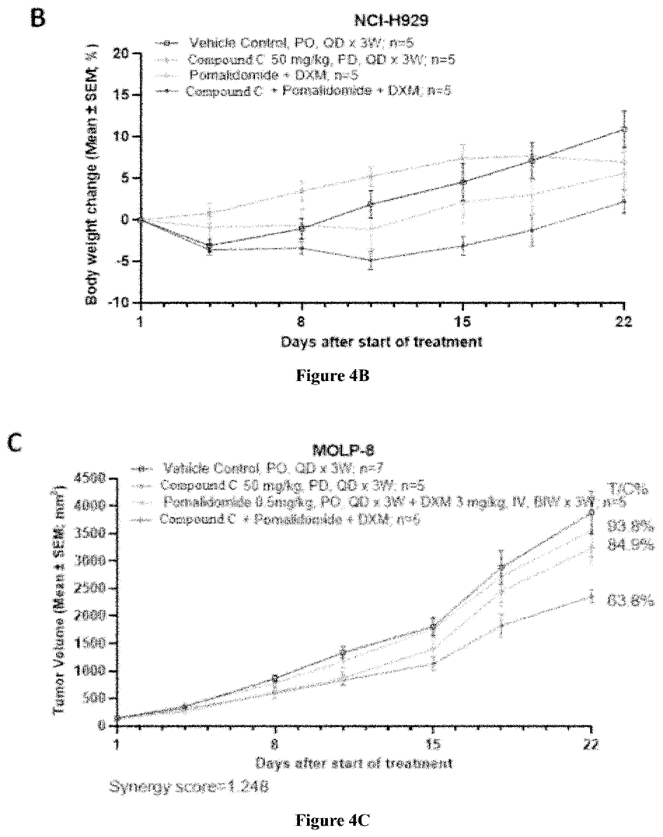

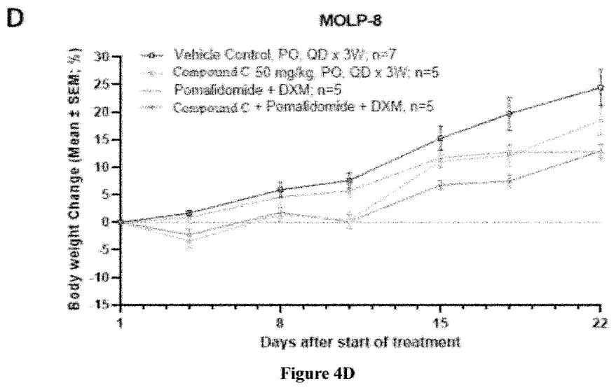

In vivo studies further revealed that coadministration of Compound C with pomalidomide enhanced tumor growth inhibition (vs either agent). Tumor growth inhibition rates of 50% in H929 and 36.2% in MOLP-8 xenograft models were also observed in response to combination treatment. FIG. 2 shows the synergistic effects of alrizomadlin and pomalidomide in MM H929 and MOLP-8 xenograft models in nude mice. (A, C) Tumor volume; (B, D) Changes in body weight (%).

1. A method for the treatment of a disease in a subject in need thereof, comprising administering to the subject a therapeutically effective amount of a compound of formula I or a pharmaceutically acceptable salt thereof; wherein the disease is acute myeloid leukemia with myelodysplasia-related changes (AML-MRC) or myelodysplastic syndrome (MDS) or multiple myeloma (MM); 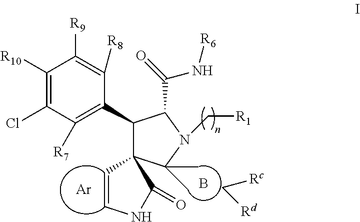
wherein, 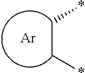
is selected from the group consisting of 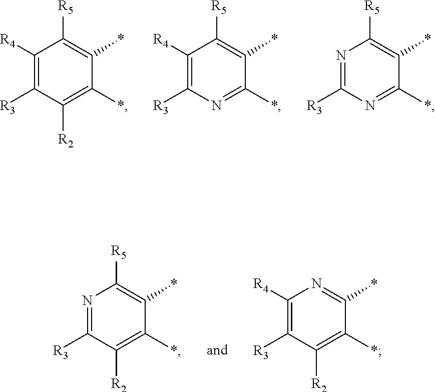
ring B is a C₄₋₇ carbocyclic ring;
R₁ is H, substituted or unsubstituted C₁₋₄ alkyl, substituted or unsubstituted C₃₋₈ cycloalkyl, substituted or unsubstituted heterocycloalkyl, OR^{a}, or NR^{a}R^{b};
n is 0, 1, or 2;
R₂, R₃, R₄, R₅, R₇, R₈, R₉, and R₁₀ are independently selected from the group consisting of H, F, Cl, CH₃ and CF₃;
R₆ is 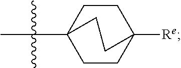
each of R^{a} is independently H, or substituted or unsubstituted C₁₋₄ alkyl;
R^{b} is H, or substituted or unsubstituted C₁₋₄ alkyl;
R^{c} and R^{d} are substituents on one carbon atom of ring B, wherein
R^{c} is H, C₁₋₃ alkyl, C₁₋₃ alkylene-OR^{a}, OR^{a}, or halogen;
R^{d} is H, C₁₋₃ alkyl, C₁₋₃ alkylene-OR^{a}, OR^{a}, or halogen;
or, R^{c} and R^{d} are taken together with the carbon to which they are attached to form a 4 to 6-membered spiro substituent optionally containing oxygen atom or nitrogen atom;
R^{e} is —C(═O)OR^{a}, —C(═O)NR^{a}R^{b}, or —C(═O)NHSO₂CH₃.

2. The method as defined in claim 1, wherein 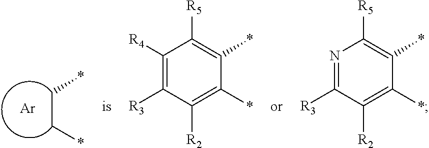
and/or, ring B is 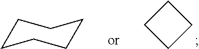
and/or, R^{c} and R^{d} are F and F; H and H; OH and CH₃; CH₃ and CH₃; CH₃ and OH; H and OH; CH₂CH₃ and CH₂CH₃; or, CH₂OH and CH₂OH;
and/or, 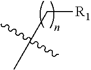
is H, CH₃, or CH₂CH₃;
and/or, R₂ is H; R₃ is halogen; and, R₄ and R₅ are H;
and/or, R₇ is halogen; each of R₈, R₉, and R₁₀ is H;
and/or, R^{e} is —C(═O)OH, —C(═O)NH₂, or —C(═O)NHSO₂CH₃.

3. The method as defined in claim 1, wherein the compound of formula (I) is selected from the group consisting of

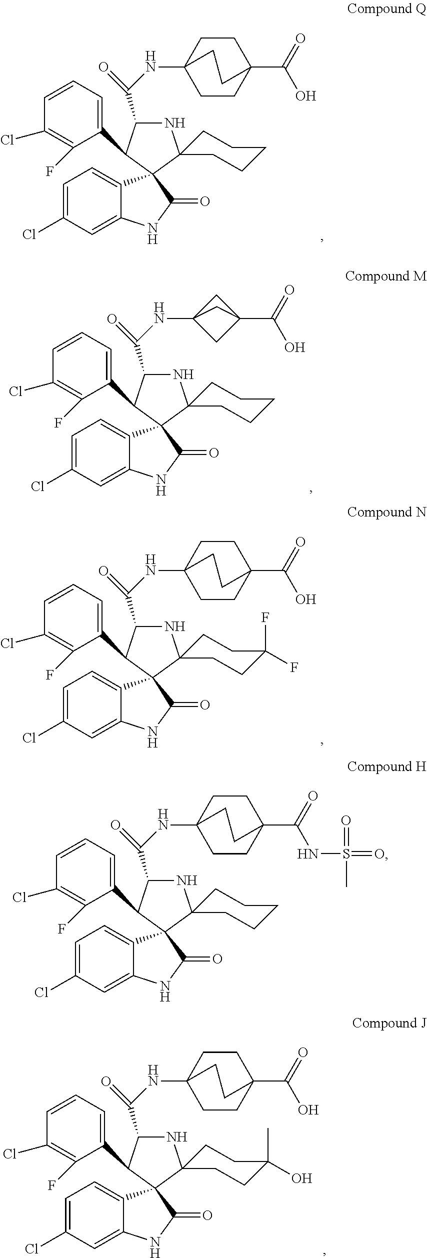 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CC(C(=O)O)(C1)C2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

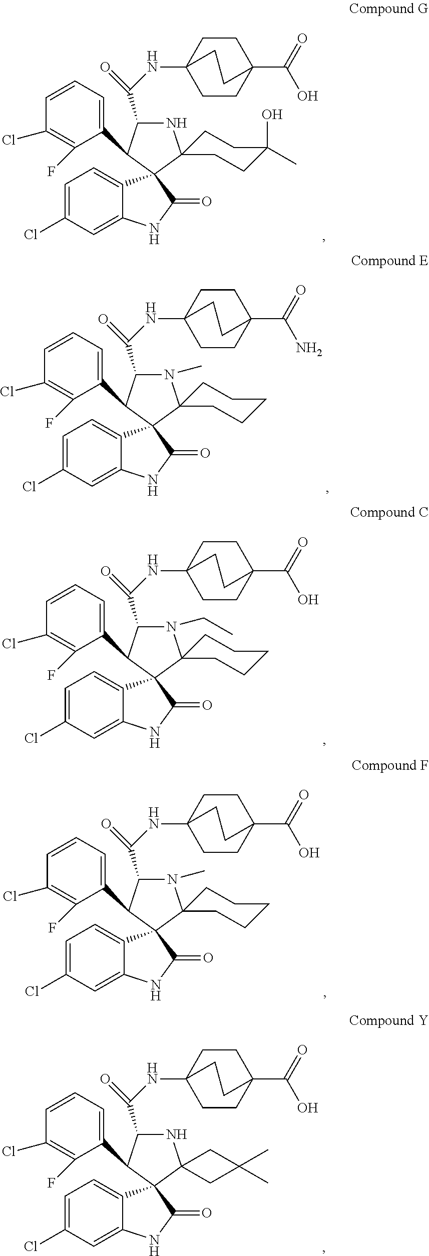 [SMILES: CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ccc21]

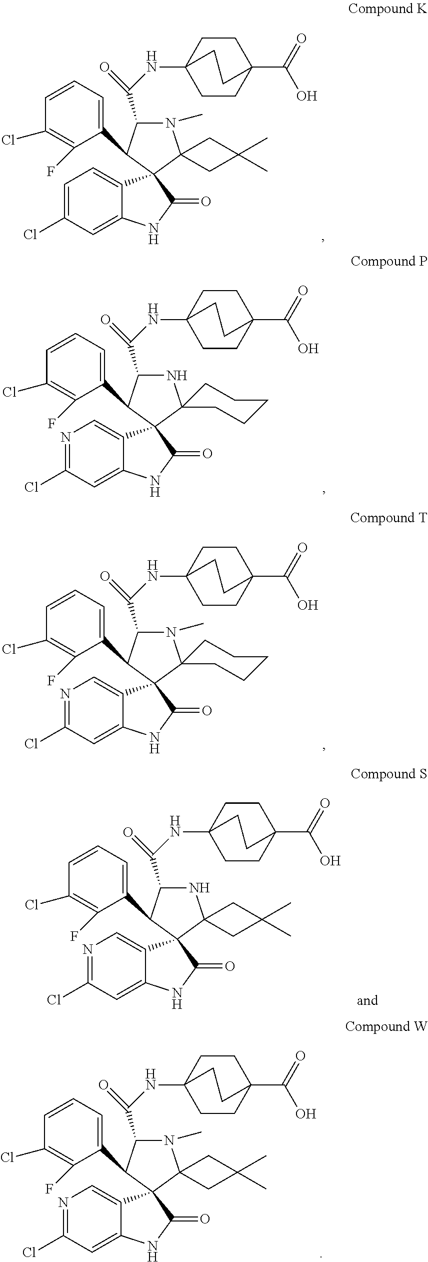 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CC(C)(C)C2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ncc21 ; CN1[C@@H](C(=O)NC23CCC(C(=O)O)(CC2)CC3)[C@H](c2cccc(Cl)c2F)[C@]2(C(=O)Nc3cc(Cl)ncc32)C12CC(C)(C)C2]

4. The method as defined in claim 1, wherein the compound of formula I is

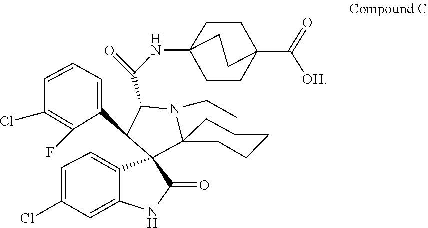 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

5. The method as defined in any one of claims 1-4, wherein the disease is AML-MRC.

6. The method as defined in any one of claims 1-5, wherein the subject to be treated has AML-MRC with no naive AML.

7. The method as defined in any one of claims 1-6, wherein the subject to be treated has AML-MRC accompanied with one or more mutations of FLT3 and IDH1/2.

8. The method as defined in any one of claims 1-7, wherein the AML-MRC is relapsed or refractory AML-MRC.

9. The method as defined in any one of claims 1-8, wherein the AML-MRC is refractory or relapse AML-MRC after treatment with one or more therapeutical agents selected from the group of azacitidine, venetoclax, decitabine, cytarabine, aclarubicin, and G-CSF.

10. The method as defined in any one of claims 1-4, wherein the disease is MDS.

11. The method as defined in any one of claims 1-4 and 10, wherein the MDS is relapsed or progressed MDS.

12. The method as defined in any one of claims 1-4 and 10-11, wherein the MDS is relapsed or progressed MDS after treatment with one or more therapeutical agents selected from the group of decitabine and lenalidomide.

13. The method as defined in any one of claims 1-12, wherein the subject is an adult.

14. The method as defined in any one of claims 1-4, wherein the disease is multiple myeloma.

15. The method as defined in any one of claims 1-14, wherein the treatment is a monotherapy with the compound of formula I or the pharmaceutically acceptable salt thereof.

16. The method as defined in any one of claims 1-15, wherein the treatment comprises at least one 28-day treatment cycle, wherein the compound of formula I or the pharmaceutically acceptable salt thereof is administered on day 1 to 5 of each treatment cycle.

17. The method as defined in any one of claims 1-15, wherein the treatment comprises at least one 28-day treatment cycle, wherein the compound of formula I or the pharmaceutically acceptable salt thereof is administered on day 1 to 7 of each treatment cycle.

18. The method as defined in any one of claims 1-13, wherein the treatment comprises administering to the subject (i) the compound of formula I or the pharmaceutically acceptable salt thereof, in combination with (ii) 5-azacitidine.

19. The method as defined in claim 18, wherein the treatment comprises at least one 28-day treatment cycle, wherein the compound of formula I or the pharmaceutically acceptable salt thereof is administered on day 1 to 5 of each treatment cycle, and the 5-azacitidine is administered on 7 out of 9 days from day 1 to day 9 of each treatment cycle.

20. The method as defined in claim 18, wherein the treatment comprises at least one 28-day treatment cycle, wherein the compound of formula I or the pharmaceutically acceptable salt thereof is administered on day 1 to 7 of each treatment cycle, and the 5-azacitidine is administered on day 1 to 7 of each treatment cycle.

21. The method as defined in any one of claims 1-14, wherein the treatment comprises administering to the subject (i) the compound of formula I or the pharmaceutically acceptable salt thereof; in combination with (ii) cytarabine.

22. The method as defined in claim 21, wherein the treatment comprises at least one 28-day treatment cycle, wherein the compound of formula I or the pharmaceutically acceptable salt thereof is administered on day 1 to 7 of each treatment cycle, and the cytarabine is administered on day 3 to 7 of each treatment cycle.

23. The method as defined in any one of claim 14, wherein the treatment comprises administering to the subject (i) the compound of formula I or the pharmaceutically acceptable salt thereof, in combination with (ii) immuno-modulatory drugs; or the treatment comprises administering to the subject (i) the compound of formula I or the pharmaceutically acceptable salt thereof, in combination with (ii) immuno-modulatory drugs and (iii) dexamethasone.

24. The method as defined in any one of claim 13, wherein the treatment comprises administering to the subject (i) the compound of formula I or the pharmaceutically acceptable salt thereof, in combination with (iii) pomalidomide, thalidomide or lenalidomide; or the treatment comprises administering to the subject (i) the compound of formula I or the pharmaceutically acceptable salt thereof, in combination with (ii) pomalidomide or thalidomide or lenalidomide and (iii) dexamethasone.

25. The method as defined in any one of claims 16, 17, 19, 20, 22, 23 and 24, wherein the 28-day treatment cycle is repeated for 1, 2, 3, 4, 5, or 6 times, or until a clinical benefit is observed.

26. The method as defined in any one of claims 1-25, wherein the compound of formula I or the pharmaceutically acceptable salt thereof is administered orally in an amount of 100 to 250 mg/day.

27. The method as defined in any one of claims 1-26, wherein the compound of formula I or the pharmaceutically acceptable salt thereof is administered orally in an amount of 100, 150, 200, or 250 mg/day.

28. The method as defined in claim 26 or 27, wherein the amount is given once a day.

29. The method as defined in any one of claims 18-20 and 25-28, wherein the 5-azacitidine is subcutaneously or intravenously injected in an amount of 75 mg/m²/day.

30. The method as defined in any one of claims 21-22 and 25-28, wherein the cytarabine is intravenously administered in an amount of 1 g/m²/day.

31. The method as defined in any one of claims 23-28, wherein the immuno-modulatory drug is administered in an amount of 0.1 to 1.0 mg/kg/day.

32. The method as defined in any one of claims 23-28, wherein the dexamethasone is subcutaneously or intravenously injected in an amount of 3 mg/kg.

33. The method as defined in any one of claims 23-28, wherein, the compound of formula I or the pharmaceutically acceptable salt thereof is administered orally in an amount of 100, 150, 200, or 250 mg/day, QD; the immuno-modulatory drug is administered orally in an amount of 0.1 to 1.0 mg/kg/day, QD; or the compound of formula I or the pharmaceutically acceptable salt thereof is administered orally in an amount of 100, 150, 200, or 250 mg/day, QD; the immuno-modulatory drug is administered orally in an amount of 0.1 to 1.0 mg/kg/day, QD, and the dexamethasone is subcutaneously or intravenously injected in an amount of 3 mg/kg, BIW; or
the compound of formula I or the pharmaceutically acceptable salt thereof is administered orally in an amount of 100, 150, 200, or 250 mg/day, QD; the immuno-modulatory drug is administered orally in an amount of 1, 1.5, 2.0, 2.5 mg/day, QD; or the compound of formula I or the pharmaceutically acceptable salt thereof is administered orally in an amount of 100, 150, 200, or 250 mg/day, QD; the immuno-modulatory drug is administered orally in an amount of 1, 1.5, 2.0, 2.5 mg/day, QD, and the dexamethasone is subcutaneously or intravenously injected in an amount of 6, 9, 12, 15 mg/day, BIW; or
the Compound C is administered orally in an amount of 4.00-4.10 mg/kg/day, QD, the pomalidomide is administered orally in an amount of 0.04-0.05 mg/kg/day, QD, or the Compound C is administered orally in an amount of 4.00-4.10 mg/kg/day, QD, the pomalidomide is administered orally in an amount of 0.04-0.05 mg/kg/day, QD, and the dexamethasone is subcutaneously or intravenously injected in an amount of 0.24-0.25 mg/kg/day, BIW.

34. A pharmaceutical combination comprising a compound of formula I and one or more anticancer reagents, wherein the formula I or the pharmaceutically acceptable salt thereof is as defined in any one of claims 1-4.

35. The pharmaceutical combination according to claim 34, wherein the anticancer reagents are selected of antimetabolite, immuno-modulatory drugs and/or dexamethasone.

36. The pharmaceutical combination according to claim 34, wherein the immuno-modulatory drugs are pomalidomide, thalidomide and lenalidomide.

37. The pharmaceutical combination according to claim 34, wherein the antimetabolite is 5-azacitidine or cytarabine.

38. The pharmaceutical combination according to any one of claims 34 to 37 for use in treating or suppressing a cancer, reducing its severity, lowering its risk or inhibiting its metastasis in an individual, wherein the cancer is preferably acute myeloid leukemia with myelodysplasia-related changes (AML-MRC) and/or myelodysplastic syndrome (MDS) and/or multiple myeloma (MM).

39. The pharmaceutical combination according to any one of claims 34 to 37, comprising a compound of formula I or the pharmaceutically acceptable salt thereof and one or more anticancer reagents,
preferably, the anticancer reagent is selected from the immuno-modulatory drugs, such as pomalidomide, thalidomide and lenalidomide, and/or dexamethasone,
preferably, the cancer is multiple myeloma (MM);
the formula I or the pharmaceutically acceptable salt thereof is as defined in any one of claims 1-4.

40. The pharmaceutical combination according to any one of claim 34, wherein, comprising (i) the compound of formula I or the pharmaceutically acceptable salt thereof and (ii) 5-azacitidine or cytarabine; wherein, the compound of formula I or the pharmaceutically acceptable salt thereof is administered orally in an amount of 100 to 250 mg/day, QD; and (ii) 5-azacitidine is subcutaneously injected in an amount of 75 mg/m²/day every day or the cytarabine is intravenously administered in an amount of 1 g/m²/day; or
wherein, comprising (i) the compound of formula I or the pharmaceutically acceptable salt thereof and (ii) the immuno-modulatory drugs, or the immuno-modulatory drugs and the dexamethasone; wherein, the compound of formula I or the pharmaceutically acceptable salt thereof is administered orally in an amount of 100 to 250 mg/day, QD; and (ii) the immuno-modulatory drugs is subcutaneously injected in an amount of 0.1 to 1.0 mg/kg/day; or the compound of formula I or the pharmaceutically acceptable salt thereof is administered orally in an amount of 100 to 250 mg/day, QD; and (ii) the immuno-modulatory drugs is subcutaneously injected in an amount of 0.1 to 1.0 mg/kg/day, and the dexamethasone is subcutaneously or intravenously injected in an amount of 3 mg/kg; or
wherein, comprising (i) the compound of formula I or the pharmaceutically acceptable salt thereof and (ii) the immuno-modulatory drugs, or the immuno-modulatory drugs and the dexamethasone; wherein, the compound of formula I or the pharmaceutically acceptable salt thereof is administered orally in an amount of 100 to 250 mg/day, QD; and (ii) the immuno-modulatory drugs is subcutaneously injected in an amount of 1 to 2.5 mg/day, QD; or the compound of formula I or the pharmaceutically acceptable salt thereof is administered orally in an amount of 100 to 250 mg/day, QD; and (ii) the immuno-modulatory drugs is subcutaneously injected in an amount of 1 to 2.5 mg/day, QD, and the dexamethasone is subcutaneously or intravenously injected in an amount of 6 to 15 mg/day, BIW; or
wherein, comprising (i) the compound of formula I or the pharmaceutically acceptable salt thereof and (ii) the immuno-modulatory drugs, or the immuno-modulatory drugs and the dexamethasone; wherein, the compound of formula I or the pharmaceutically acceptable salt thereof is administered orally in an amount of 4.00-4.10 mg/kg/day, QD; and (ii) the immuno-modulatory drugs is subcutaneously injected in an amount of 0.04-0.05 mg/kg/day, QD; or the compound of formula I or the pharmaceutically acceptable salt thereof is administered orally in an amount of 4.00-4.10 mg/kg/day, QD; and (ii) the immuno-modulatory drugs is subcutaneously injected in an amount of 0.04-0.05 mg/kg/day, and the dexamethasone is subcutaneously or intravenously injected in an amount of 0.24-0.25 mg/kg/day, BIW.

41. A pharmaceutical composition, comprising (i) the compound of formula I or a pharmaceutically acceptable salt thereof and (ii) immuno-modulatory drugs (e.g., pomalidomide, thalidomide or lenalidomide), wherein, the mass ratio of (i) the compound of formula I or a pharmaceutically acceptable salt thereof/(ii) immuno-modulatory drugs is 100:1-1:100; such as comprising (i) the amount of the compound of formula I or the pharmaceutically acceptable salt thereof is 100, 150, 200, 250 mg and (ii) the amount of the immuno-modulatory drugs is 1, 1.5, 2.0, 2.5 mg.
